(19) United States
(12) Patent Application Publication
(10) Pub. No.: US 2026/0262695 A1
(43) Pub. Date: Sep. 10, 2026

(54) NUTRITIONAL COMPOSITIONS AND METHODS FOR OPTIMIZING PROTEIN QUALITY IN PRODUCTS CONTAINING PLANT PROTEIN SOURCES

(71) Applicant: SOCIÉTÉ DES PRODUITS NESTLÉ S.A., Vevey (CH)

(72) Inventors: Maryann ROLANDS, Wallisellen (CH); Kim-Anne LE BUR, Lausanne 26 (CH); Fabio MAINARDI, Lausanne (CH)

(21) Appl. No.: 19/165,673

(22) PCT Filed: Mar. 14, 2024
(86) PCT No.: PCT/EP2024/056763

§ 371 Date: Sep. 16, 2025

Related U.S. Application Data
(60) Provisional application No. 63/452,805, filed on Mar. 17, 2023.

(51) Int. Cl. A23L 33/185; A23L 33/16; A23L 33/175; A23L 33/21; G16H 20/60

(52) U.S. Cl. CPC A23L 33/185; A23L 33/16; A23L 33/175; A23L 33/21; G16H 20/60

(57) ABSTRACT

A method of manufacturing a composition having optimal protein quality includes determining a ratio of a first protein source to a second protein source with an optimal protein quality using an optimization model and manufacturing a composition with that ratio of protein sources. Preferably, the composition has a PDCAAS score of about 1. The composition can have protein sources with limiting amino acids and preferably different protein sources with different limiting amino acids. The composition may be administered to an individual to increase protein bioavailability of the protein consumed by the individual. Preferably, the individual consumes a plant based diet.

### TECHNICAL FIELD

The present disclosure generally relates to compositions and methods for optimizing the protein quality of products containing plant protein sources.

### BACKGROUND

The popularity of vegetarian and vegan diets has steadily increased during the past decade. For example, studies in France, Netherlands, Sweden, Germany, and the US reported higher prevalence rates for vegetarians, ranging from 1.7% to 5.4%. Diets incorporating more plant-based foods and less animal-derived products, such as the portfolio diet, the Mediterranean diet, and the DASH diet, have been associated with a reduced risk of cardiovascular diseases, metabolic syndrome, type 2 diabetes, and some cancers. One of the postulated mechanisms for these beneficial effects lies in the nutritional composition of plant-based foods. Specifically, plant-based diets are traditionally lower in energy density, with higher unsaturated fats, dietary fibers, vitamins, minerals and some phytonutrients, which have shown protective effects against chronic diseases.

A recent large US prospective cohort showed a higher plant protein intake was associated with lower risk of overall mortality and cardiovascular disease mortality. Another study found those with higher animal protein intake and a higher animal-to-plant protein ratio had higher cardiometabolic risk factors, such as higher waist circumference and fasting blood glucose. The intake of plant proteins was associated with lower systolic and diastolic blood pressure and the consumption of soy protein with isoflavones resulted in lower total and LDL cholesterol than did the consumption of animal protein, except for whey isolate. However, plant-based diets also include sub-optimal protein quantity and quality, low intakes of long-chain omega-3 fatty acids, vitamin B12, vitamin D, calcium, iodine, selenium, iron and zinc.

Proteins are made up of amino acids and play several roles in normal body functions such as growth, maintenance, biochemical reactions, chemical messengers, transport proteins, fluid balance, immune function support, muscle support, regulation, and expression of DNA and RNA. The European Food Safety Authorities recommend that adults consume at least 0.83 g of protein per kg body weight per day and adults in the US are recommended to consume 10-35% total energy from protein. With current dietary recommendations focused on protein quantity, there is a lack of insight into the protein quality of diets. Most plant proteins contain all 20 amino acids but with a limited amount of certain essential amino acids, known as their limiting amino acid. Therefore, for individuals relying mostly on plant protein sources, a high-quality protein intake can only be achieved by specific combinations of plant protein sources, allowing to compensate for the various limiting amino acids.

### SUMMARY

Accordingly, an aspect of the present disclosure is a method of identifying an optimal ratio of a first protein source to a second protein source, by (i) receiving a first list of ingredients for the first protein source and receiving a second list of ingredients for the second protein source, (ii) computing a plurality of combinations of the ingredients from the first list of ingredients with the ingredients from the second list of ingredients (optionally, all possible combinations), (iii) computing a protein quality score for each combination at a plurality of ratios, preferably every integer ratio, and (iv) identifying the optimal ratio of the first protein source to the second protein source, wherein the optimal ratio is a ratio of the first protein source to the second protein source meets or exceeds a predefined protein quality score.

Another aspect of the present disclosure is a method of manufacturing a composition having optimal protein quality. The method comprises determining an optimal ratio of a first protein source to a second protein source, by (i) receiving a first list of ingredients for the first protein source and receiving a second list of ingredients for the second protein source, (ii) computing a plurality of combinations of the ingredients from the first list of ingredients with the ingredients from the second list of ingredients (optionally, all possible combinations), (iii) computing a protein quality score for each combination at a plurality of ratios, preferably every integer ratio, and (iv) identifying the optimal ratio of the first protein source to the second protein source, wherein the optimal ratio is a ratio of the first protein source to the second protein source meets or exceeds a predefined protein quality score. The method also includes combining the first protein source and the second protein source at the optimal ratio to thereby form at least a portion of the composition. The method optionally includes adding, to the first and second protein sources, one or more additional components such as carbohydrates, fat, vitamins, minerals, or a third protein source.

In some embodiments, the protein quality score is a PDCAAS score of about 1. In some embodiments, the first protein source comprises a limiting amino acid; and the second protein source comprises a limiting amino acid different than the limiting amino acid of the first protein source.

Other aspects of the present disclosure are a composition with optimal protein quality (e.g., a composition made by any method disclosed herein) and a method of increasing protein digestibility, protein absorption, and protein bioavailability (e.g., by administering a composition made by any method disclosed herein). The composition includes a first protein source and a second protein source. The first protein source is selected from the group consisting of a protein with a lysine limiting amino acid and a protein with no limiting amino acid; and the second protein source is selected from the group consisting of a protein with a lysine limiting amino acid, a protein with methionine and cysteine limiting amino acid, and a protein with no limiting amino acid. In some embodiments, the first protein source is at least about 10% of the combination of the first and second protein sources, and optionally a third protein source.

In some embodiments, the composition optionally includes one or more additional components such as carbohydrates, fat, vitamins, minerals, or a third protein source, in addition to the first and second protein sources.

In some embodiments, the first protein source comprises a protein with a lysine limiting amino acid; and the protein with the lysine limiting amino acid comprises at least one protein from one or more of cereals, nuts, or seeds.

In some embodiments, the second protein source comprises a protein with a methionine and cysteine limiting amino acid; and the protein with the methionine and cysteine limiting amino acid comprises at least one protein from legumes.

In some embodiments, the first protein source is plant based and comprises a protein with no limiting amino acids.

In some embodiments, the composition optionally further includes one or more additional protein sources, for example a third protein source. The third protein source may be selected from the group consisting of a protein with a lysine limiting amino acid, a protein with methionine and cysteine limiting amino acid, and a protein with no limiting amino acid; and the third protein source is a different protein source than the first and second protein sources. In other embodiments, the first and second protein sources, and optionally the third protein source, in the composition are the only proteins in the composition.

Yet another aspect of the present disclosure is a composition comprising a protein source, the protein source comprising a combination of at least 10% of a protein with a lysine limiting amino acid, 10-60% of a protein with methionine and cysteine limiting amino acids, and 30-80% of a plant-based protein with no limiting amino acid, wherein the protein source comprises a PDCAAS score of about 1.

In some embodiments, the protein source comprises 95-300 kcal., 3.2-10% of the protein source, 1.5-5 grams of fiber, 0.9-2.8 mg iron, 79-250 mg calcium, and/or 0.1-1.2 mg zinc.

In some embodiments, the protein with a lysine limiting amino acid is selected from the group consisting of oat, barley, seitan, pumpkin seeds, white rice, and combinations thereof.

In some embodiments, the protein with methionine and cysteine limiting amino acids is selected from the group consisting of kidney bean, potato, lentils, peas split, fava bean, black beans, peas, and combinations thereof.

In some embodiments, the protein with no limiting amino acid is selected from the group consisting of soy milk, buckwheat, tofu, and combinations thereof.

In a preferred embodiment, the protein source comprises 50-60% of the protein with methionine and cysteine limiting amino acids and/or the protein source comprises 30-40% of the plant-based protein with no limiting amino acid.

In some embodiments, the protein source with lysine as the limiting amino acid, the protein source with methionine and cysteine as the limiting amino acids, and the protein source without any limiting amino acids are the only protein sources in the composition. In some embodiments, the composition is plant based.

One or more embodiments disclosed herein advantageously determine optimal protein quality in a combination of protein sources to thereby provide higher digestibility, absorption, and/or bioavailability of protein to an individual.

Another advantage of the present disclosure is a composition for individuals on a plant based diet, the composition having known protein quality to ensure the individuals receive a sufficient amount of protein in their diet.

Additional features and advantages are described herein and will be apparent from the following Figures and Detailed Description.

### BRIEF DESCRIPTION OF THE FIGURES

FIG. 1 shows an example optimization model to determine optimal protein quality from mixed protein sources, in an embodiment according to the present disclosure.

FIG. 2 shows boxplots of the extreme and equal ratio from each ingredient group of limiting amino acids, in an embodiment according to the present disclosure.

FIG. 3 shows boxplots of PDCAAS values for ingredient mixes where the ingredient mix comprises 10% of a protein source without any limiting amino acids, in an embodiment according to the present disclosure.

FIG. 4 shows boxplots of PDCAAS values for ingredient mixes where the ingredient mix comprises 20% of a protein source without any limiting amino acids, in an embodiment according to the present disclosure.

FIG. 5 shows boxplots of PDCAAS values for ingredient mixes where the ingredient mix comprises 30% of a protein source without any limiting amino acids, in an embodiment according to the present disclosure.

FIG. 6 shows boxplots of PDCAAS values for ingredient mixes containing protein sources from cereal/nuts and legumes at different ratios, in an embodiment according to the present disclosure.

FIG. 7 shows boxplots of PDCAAS values for ingredient mixes where the ingredient mix comprises 30% of a protein source with lysine as the limiting amino acid, in an embodiment according to the present disclosure.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages disclosed and claimed herein are by weight of the total weight of the composition unless expressed otherwise. As used herein, “about,” “approximately” and “substantially” are understood to refer to numbers in a range of numerals, for example the range of −25% to +25% of the referenced number, preferably −10% to +10% of the referenced number, more preferably −5% to +5% of the referenced number, most preferably −1% to 1% of the referenced number. A range described as “between” two values includes those endpoint values. All numerical ranges herein include all integers, whole or tractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a protein” or “the protein” includes two or more proteins.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions and methods disclosed herein may lack any element that is not specifically disclosed herein. Thus, a disclosure of an embodiment using the term “comprising” includes a disclosure of embodiments “consisting essentially of” and “consisting of” the components or steps identified.

The terms “at least one of” and “and/or” used respectively in the context of “at least one of X and Y” and “X and/or Y” should be interpreted as “X without Y,” or “Y without X,” or “both X and Y.” Where used herein, the terms “example” and “such as,” particularly when followed by a listing of terms, are merely exemplary and illustrative and should not be deemed to be exclusive or comprehensive.

The term “composition” may mean a food, beverage, dietary supplement, complete nutrition or oral nutritional supplement (ONS) or medical food composition, or mixture thereof.

The terms “food,” “food supplement,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a supplement.

Within the context of the present disclosure, the term “beverage,” “beverage product” and “beverage composition” mean a potable liquid product or composition for ingestion by an individual such as a human and provides water and may also include one or more nutrients and other ingredients safe for human consumption to the individual. The compositions of the present disclosure, including the many embodiments described herein, can comprise, consist of, or consist essentially of one or more of the nutrients listed above, as well as any additional or optional ingredients, components safe for human consumption and otherwise useful in a diet.

Within the context of the present disclosure, “dietary supplements” are products taken by mouth that contain one or more dietary ingredient, such as vitamins, minerals, amino acids, fatty acids, fibers and/or herbs and other botanical ingredients used to supplement the diet. Dietary supplements come in many forms and may be available as tablets, capsules, powders, liquids, and formulated into specific foods, such as “energy” bars.

As used herein, “optimal protein quality” shall refer to a combination of protein sources with a protein quality score above or meeting a predefined protein quality score. In a preferred embodiment, “optimal protein quality” is a combination of protein sources with a PDCAAS score of about 1. In another embodiment, “optimal protein quality” is a combination of protein sources with a DIAAS score at or above about 1.

As used herein, “every integer ratio” shall refer to each integer ratio from 1:99 to 99:1.

The term “protein bioavailability” shall refer to how well the body utilizes a specific type of protein. A protein is considered highly bioavailable if it is easy to digest, absorb and make into other proteins.

“IAAs,” “Indispensable Amino Acids,” or “Essential Amino Acids” shall refer to amino acids that cannot be synthesized by the body, but instead are only obtained from dietary intake. Adequate amounts of IAAs in the diet are required to meet the metabolic demand and to assure proper functioning of the human body.

The term “Protein Digestibility Corrected Amino Acids Score” or “PDCAAS” is the ratio of the limiting indigestible amino acid in test protein compared to reference protein corrected for faecal protein digestibility.

As used herein “Digestible Indispensable Amino Acid Score” or “DIAAS” is a metric that produces a score for each indispensable amino acid based on the concentration of each digestible indispensable amino acid, calculated from the concentration of each indispensable amino acid (IAA) (per g protein) and its ileal digestibility. For each IAA, this value is then compared with a reference pattern for each age group (“>3 years or 6 months-3 years”) and the IAA with lowest score relative to the reference pattern is the first limiting IAA.

The term “plant-based” shall refer to products which typically comprise vegetables, fruits, tubers, legumes, cereals, seeds, oilseeds and/or nuts and are products not derived from animal products.

“Limiting amino acid” as used herein shall refer to the amino acids deficient in a certain source. Without this limiting amino acid, protein synthesis is limited.

The term “cereals” includes wheat, rice, maize, barley, sorghum, millet, oats, rye, triticale, fonio, and pseudocereals (e.g. amaranth, breadut, buckwheat, chia, cockscomb, pitseed goosefoot, qaniwa, quinoa, and wattle seed).

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual.

### EMBODIMENTS

In a first aspect of the present disclosure, a method of manufacturing a composition having optimal protein quality comprises determining a ratio of a first protein source to a second protein source, the ratio having optimal protein quality and determined using an optimization model; and combining the first protein source and the second protein source at the ratio to thereby form at least a portion of the composition. In some embodiments, the method includes determining a ratio of a first protein source, a second protein source, and one or more additional protein sources. The method optionally includes adding, to the first and second protein sources, one or more additional components such as carbohydrates, fat, vitamins, minerals, or a third protein source.

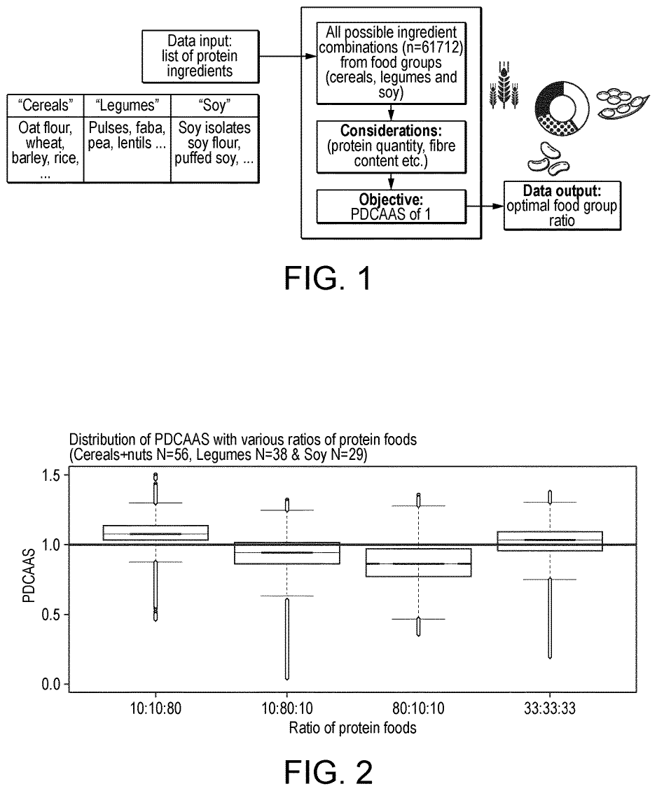

The optimization model of the present disclosure may be run on a processor, such as a computer. A schematic of the optimization model is displayed in FIG. 1. A list of protein ingredients grouped by category, such as grouped by the protein's most limiting amino acid or grouped by the individual protein quality levels, is input into the optimization model to determine a protein mix with an optimal protein quality. The model computes and determines a plurality of possible ingredient combinations for each group (optionally, all possible ingredient combinations for each group). For example, if the categories are cereals, legumes, and soy and the ingredients for cereal are oat flour and wheat, the ingredients for legumes are pulses and faba beans, and the ingredients for soy are soy isolates and soy flour, the model will determine a plurality of possible combinations of the cereal, legumes, and soy ingredients (optionally, all possible combinations of the cereal, legumes, and soy ingredients). In this specific example, the simulation will determine a combination comprising oat flour, pulses, and soy isolates, a combination comprising wheat, pulses, and soy isolates, a combination comprising oat flour, faba beans, and soy isolates, and so on.

The model then determines a protein quality score for each ingredient combination at each ratio or a set range of ratios. In some embodiments, the protein quality score takes into account amino acid score and protein digestibility. A protein combination with optimal protein quality is a combination with a ratio where the protein quality score of the combination exceeds or meets a certain predefined value. In some embodiments, the protein quality score may be a PDCAAS score. For example, if the protein quality score is PDCAAS, then an optimal protein quality is a protein combination with a PDCAAS score of about 1. In some embodiments, the protein quality score may be a DIAAS score. In some embodiments, the optimal protein quality is a protein combination with a DIAAS score meeting or exceeding about 1. Additionally, one skilled in the art would understand that alternative protein quality scores and predefined values to assess optimal protein quality could be used.

When determining an optimal ratio of protein sources, the model may take into account different variables treated as constraints, such as protein quantity, fiber content, type of product to be manufactured, cost, sustainability, technical feasibility, and nutritional information. Nutritional information may include vitamin, mineral, carbohydrate, and lipid levels. In a preferred embodiment, the model takes into account one or more of energy amount, protein amount, fiber amount, iron amount, calcium amount, and zinc amount. The variables may be selected in some models and not in others. In some embodiments, the final output of the model is different ratios of protein groups that provide optimal protein quality.

In some embodiments, the protein ingredients are grouped by the protein's most limiting amino acid group, preferably into a protein source with lysine as the limiting amino acid, a protein source with methionine and cysteine as the limiting amino acids, and an additional protein source without any limiting amino acids. In this embodiment, the model may be further constrained to ensure optimal protein mixes between the groups. In some embodiments, a condition of the model includes at least 10% coming from each of the three groups. In some embodiments, a condition of the model includes from 10% to 80% of the group with no limiting amino acids in the protein combination.

The method may comprise manufacturing a product, such as a food or dietary supplement, a food product, a food composition, or a beverage, with the ratio of proteins from the optimization model to thereby create a product with optimal protein quality. The product may further comprise one or more additional components such as carbohydrates, fat, vitamins or minerals. The product may be suitable for oral consumption by an individual who is vegan, flexitarian or vegetarian. Additionally, the product may be suitable for oral consumption by an individual planning to switch to a vegan, flexitarian, or plant based diet. For example, the composition may be essentially free of meat, dairy products, and eggs. In some embodiments, the composition may contain a hybrid of plant based and animal based ingredients, such as dairy or eggs.

In another aspect of the present disclosure, a composition with optimal protein quality comprises a first protein source and a second protein source. Some embodiments of the composition have a ratio of the first protein source to the second protein source that produces optimal protein quality and high absorption, digestibility, and/or bioavailability of the protein in the body. This ratio may be determined using the optimization model disclosed herein. In another aspect, a method for increasing at least one of protein absorption, protein digestibility, and protein bioavailability comprises providing the composition to an individual and/or orally administering the composition to an individual. Preferably, the individual may be an individual with a plant based diet (e.g., vegan, vegetarian, or flexitarian).

In some embodiments, the first and second protein sources are protein sources with one or more limiting amino acids. The limiting amino acid may be an essential amino acid, also known as an indispensable amino acid. Essential amino acids include lysine, methionine, cysteine, histidine, isoleucine, leucine, phenylalanine, tryptophan, threonine, and valine. In some embodiments, one or both of the first and second protein sources are plant based.

Preferably, the limiting amino acid of the first protein is different from the limiting amino acid of the second protein. This embodiment results in one protein source compensating for, or otherwise mitigating, the deficiency of the limiting amino acid in the other protein source and vice versa. Even more preferably, the limiting amino acid of the first protein source is lysine and the limiting amino acids of the second protein source are methionine and cysteine.

Lysine is the limiting amino acid of proteins derived from most cereals, nuts, and seeds. This means that the majority of proteins derived from cereals, nuts, and seeds do not contain enough lysine for protein synthesis in the body. Examples of protein sources from cereals, nuts, and seeds include flax seeds, cashew nuts, chia seeds, white rice, etc. Protein sources with limited amounts of methionine and cysteine include legumes. Legumes include, but are not limited to, faba beans, pea protein isolates, pea protein concentrate, split peas, adzuki beans, etc.

In some embodiments, the first protein source or the second protein source is a protein source with no limiting amino acids. A protein source with no limiting amino acids has all of the essential proteins needed for protein synthesis in the body. Preferably, the protein source with no limiting amino acids is a plant based protein source, for example, a protein source derived from soy. The protein source derived from soy may be raw mature soybeans, black soybeans, soy protein flour, soybean protein isolate, etc. In some embodiments, the protein source with no limiting amino acids is an animal derived protein source, such as protein from dairy or eggs.

In some embodiments, the first protein source or the second protein source is a protein derived from legumes. For example, the protein may be derived from soy, faba beans, lentils, split peas, pinto beans, kidney beans, lima beans, adzuki beans, chickpeas, kidney beans, and combination thereof.

The composition may comprise one or more additional protein sources beyond the first and second protein sources, for example a third protein source or a fourth protein source. The one or more additional protein sources may include one or more limiting amino acids. The additional protein sources may have no limiting amino acids. All of the protein sources may be in the composition in a ratio so that the combination of protein sources has optimal protein quality. In some embodiments, optimal protein quality is indicated by a PDCAAS score of about 1. In some embodiments, at least one protein source is a plant-based protein source and at least one protein source is an animal protein source, such as protein from dairy or eggs. In some embodiments, all of the protein sources are plant-based.

In a preferred embodiment, the first protein source includes a protein source with lysine as the limiting amino acid. In this embodiment, the ratio of the lysine limited protein to the second protein source is preferably between about 20:80 and about 40:60, between about 20:80 and about 25:75, between about 25:75 and about 30:70, between about 30:70 and about 35:65, or between 35:65 and about 40:60. Additionally or alternatively, the protein source with lysine as the limiting amino acid is preferably at least 10%, at least 15% at least 20%, at least 25%, at least 30%, at least 35%, or at least 40% of the combination of the first and second protein sources, and optionally the third protein source. Preferably, the second protein source comprises legumes.

In another preferred embodiment, the composition includes a combination of protein sources including a protein source with lysine as the limiting amino acid, a protein source with methionine and cysteine as the limiting amino acids, and an additional protein source without any limiting amino acids. In this embodiment, the protein source without any limiting amino acids is at least 20%, at least 25%, at least 30%, at least 35%, or at least 40% of the combination of the protein sources, such as the combination of the first, second, and third protein sources. In some embodiments, the protein source without any limiting amino acids is between about 30% and about 80%, preferably between about 30% and about 40%, of the combination of protein sources. In some embodiments, the protein source with methionine and cysteine as the limiting amino acids is between about 10% and about 60%, preferably between about 50% and about 60%, of the combination of protein sources. In some embodiments, the protein source with lysine as the limiting amino acid is at least 10% of the combination of protein sources. In some embodiments, the combination of protein sources is the only protein source in the composition.

The composition or the combination of protein sources can further include one or more micro and macro nutrient limitations, such as levels of energy, protein, fiber, iron, calcium, and/or zinc. In some embodiments, the composition or the combination of protein sources can be limited with nutrient constraints beneficial for mitigating deficiencies in typical plant based diets. For example, the composition or combination of protein sources may include energy ranging from about 90-500 kcal, protein ranging from about 3-10%, and/or dietary fiber ranging from about 1.5-5 g. Additionally or alternatively, the composition or combination of protein sources includes iron ranging from about 0.9-2.8 g, calcium ranging from about 79-250 mg, and/or zinc ranging from about 0.1-1.2 g. The composition can be in any oral nutritional form, e.g., as a health drink, as a ready-made drink, optionally as a soft drink, including juices, coffee, tea, milk-shake, yogurt drink, smoothie or soy-based drink; in a food bar; a powder, such as in sachet form; or dispersed in foods of any sort, such as baked products, cereal bars, dairy bars, snack-foods, soups, breakfast cereals, muesli, candies, tabs, cookies, biscuits, crackers (such as rice crackers), and dairy and plant-based products.

The composition can be administered to the individual by at least one route selected from the group consisting of oral, topical, enteral and parenteral. For example, the protein sources can be administered in a composition selected from the group consisting of a nutritionally complete product, a drink, a dietary supplement, a meal replacement, a food additive, a supplement to a food product, a powder for dissolution, an enteral nutrition product, an infant formula, a capsule, and combinations thereof. The combination is preferably orally administered in a composition such as a food composition.

The composition can be administered to the individual at least two days per week, at least three days per week, all seven days of the week respecting serving size; for at least one week, at least one month, at least two months, at least three months, at least six months, or even longer. In some embodiments, the composition is administered to the individual consecutively for a number of days. In an embodiment, the composition can be administered to the individual daily for at least 30, 60 or 90 consecutive days.

### EXAMPLES

### Example 1

In order to determine ratios of proteins for optimal protein quality, a model was used that determined the PDCAAS score for different ratios of proteins using different ingredient mixes. First, food groups with the most limiting amino acids were determined. In this example, lysine limiting foods, which encompass cereals, seeds, and nuts, methionine and cysteine limiting foods, which encompass legumes, and plant based proteins with no limiting amino acids, which encompass soy, were selected. Next, we generated a list of ingredients from each food group. Samples of different ingredients for each food group are shown in Table 1. The lysine limiting group contained 56 different ingredients, the methionine and cysteine group contained 38 different ingredients, the no limiting amino acid group contained 29 different ingredients.

TABLE 1
List of Example Ingredients for Each Protein Group

| “Lysine limiting” | | |
|---|---|---|
| (e.g., cereals, nuts, seeds, | “Methionine + cysteine limiting” | “No limiting amino acids” |
| etc.) | (e.g., legumes, etc.) | (e.g., soy, etc.) |
| (N = 56) | (N = 38) | (N = 29) |
| Flax seeds | Faba bean raw | Soybean (raw mature seeds, USDA) |
| Cashew nuts | Pea protein concentrate/isolate | Black soybean |
| Chia seeds | Split peas | Soy protein flour, 40% protein |
| | | (Dehulled) |
| White rice | Adzuki beans | Soybean protein isolate D150L |
| | | (Wilpro) |
| . . . | . . . | . . . |

The different ingredients of the three food groups were input into the model, where the model generated all combinations of ingredients at different ratios. The model uncovered 61,712 different mixes of ingredients for each ratio. A distribution of the PDCAAS values of each mix at each ratio were plotted to determine if the ratios of the three groups had an average PDCAAS score of over 1, indicating that the combination of proteins had optimal protein quality.

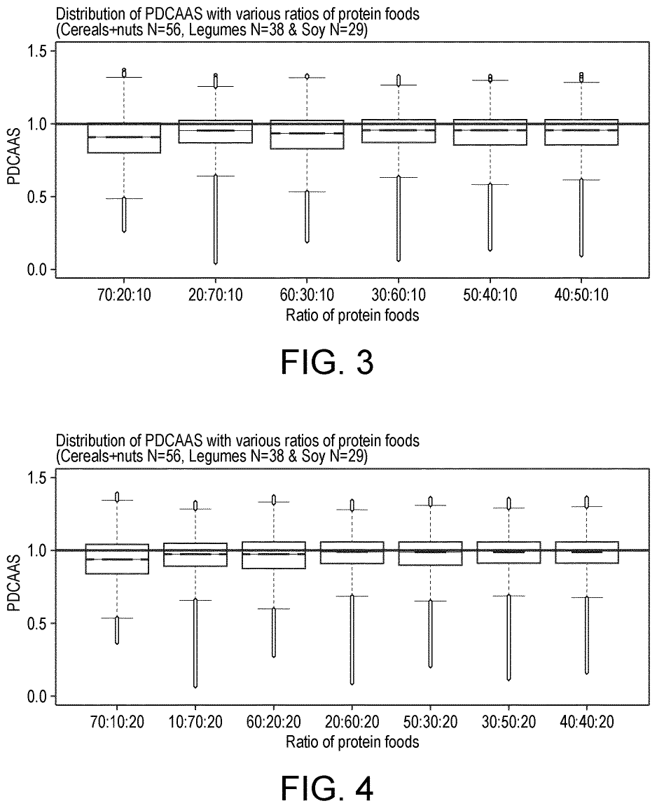

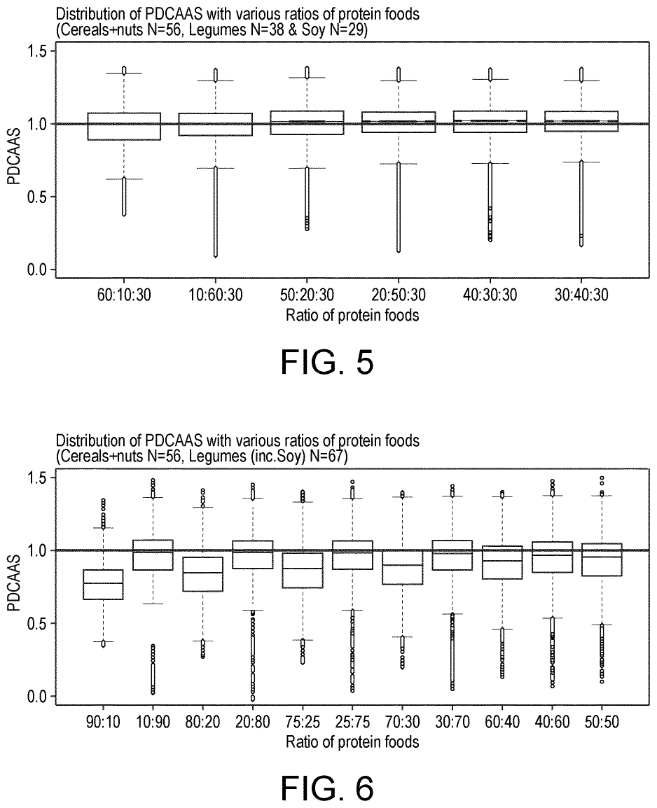

FIGS. 2-5 show example plots of the distribution of PDCAAS scores for the three protein groups at different ratios. In FIGS. 3-5, the amount of soy was slightly increased from 10%, to 20%, to 30%. The results of the model indicated the ingredient mix with at least 30% soy typically provide a PDCAAS score of over 1.

### Example 2

The protein quality of a combination of two protein sources, cereals and legumes, was assessed at different ratios. Protein ratios of current products are shown in Table 2. No recipe has ingredients from all three sources of protein. It was then decided to further investigate two groups of protein sources for optimal protein quality on a product level.

TABLE 2
Examples of Protein Ratios of Different Products

| Current products | “Cereals” | “Legumes” | “Soy” |
|---|---|---|---|
| Plant-based beverage | 18 | 82 | 0 |
| Meat alternative A | 0 | 0 | 96 |
| Meat alternative B | 40 | 0 | 52 |
| Fish alternative | 29 | 66 | 0 |
| Falafel | 0 | 78 | 0 |
| Average ratios | 28 | 80 | 67 |

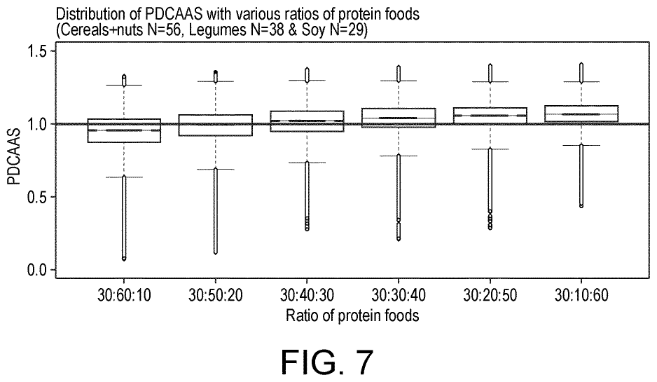

FIG. 6 shows a boxplot of PDCAAS scores of ingredient mixes with different ratios of cereals and legumes. The protein mixes with about 20-40% of cereals and about 60-80% of legumes displayed the best protein quality. The model was run with 30% cereal and different varying ratios of legumes and soy, which confirmed that protein quality of the ingredient mix was optimal with at least 30% cereal protein, as shown in FIG. 7.

### Example 3

The protein quality of a combination of three proteins was assessed according to the model of FIG. 1. In this example, the following list of ingredients from each protein group was input in the model to determine optimal ratios between groups and examples of optimal mixes.

TABLE 3
List of Ingredients for Each Protein Group

| No-limiting (N = 7) | Lysine (N = 26) | Methionine cysteine (N = 18) |
|---|---|---|
| Buckwheat groats roasted, | Barley pearl cooked | Beans, kidney, all types, mature |
| cooked | | seeds, cooked, boiled, without salt |
| Cowpeas, catjang, mature | Bread, pita, white, | Beans, adzuki, mature seeds, |
| seeds, cooked, boiled, | unenriched | cooked, boiled, without salt |
| without salt | | |
| Tofu | Almond butter | Beans, black, mature seeds, cooked, |
| | | boiled, without salt |
| Soybeans, mature cooked, | Almonds raw | Chickpeas (garbanzo beans, bengal |
| boiled, without salt | | gram), mature seeds, cooked, |
| | | boiled, without salt |
| Tofu, fried | Nuts, cashew butter, plain, | Broad beans (fava beans), mature |
| | without salt added | seeds, cooked, boiled, without salt |
| Soya milk | Nuts, cashew nuts, raw | Peas, split, mature seeds, cooked, |
| | | boiled, without salt |
| Yeast (Saccharomyces | Seeds, chia seeds, dried | Lupins, mature seeds, cooked, |
| cerevisiea) | | boiled, without salt |
| | Nuts, coconut milk, canned | Mung beans, mature seeds, cooked, |
| | (liquid expressed from | boiled, without salt |
| | grated meat and water) | |
| | Millet, cooked | Beans, navy, mature seeds, cooked, |
| | | boiled, without salt |
| | Oat | Beans, pinto, mature seeds, cooked, |
| | | boiled, without salt |
| | Oat bran, raw | Potatoes, boiled, cooked without |
| | | skin, flesh, without salt |
| | Oat bran, cooked | Lentils, mature seeds, cooked, |
| | | boiled, without salt |
| | Peanuts, all types, raw | Yellow peas, split, cooked, boiled |
| | Pumpkin and squash seed | Mushrooms, portabella, raw |
| | kernels, dried | |
| | Rice, brown, long-grain, | Carrots, cooked, boiled, drained, |
| | cooked | without salt |
| | Rice, white, long-grain, | Apples, raw, golden delicious, with |
| | regular, unenriched, | skin |
| | cooked without salt | |
| | Seeds, sesame seed | Kale, cooked, boiled, drained, |
| | kernels, toasted, without | without salt |
| | salt added (decorticated) | |
| | Sunflower seed kernels, | Potatoes, french fried, cottage-cut, |
| | dried | salt not added in processing, frozen, |
| | | oven-heated |
| | Nuts, walnuts, black, dried | |
| | Walnuts, English | |
| | Seeds, watermelon seed | |
| | kernels, dried | |
| | Pasta, whole-wheat, | |
| | cooked | |
| | Fried seitan coated with | |
| | wheat flour | |
| | Tortillas, ready-to-bake | |
| | or -fry, corn | |
| | Bread, whole-wheat, | |
| | prepared from recipe | |
| | Cereals ready-to-eat, Corn | |
| | Flakes | |

Certain nutritional constraints were considered in the model, including calorie content, quantity of protein, and amounts of fiber, iron, calcium, and/or zinc. In this specific example, other conditions were set to ensure a variety of consumption from protein sources between them. For example, the model ensured at least 10% should come from each of the three limiting amino acid sources so no source has 0% in the final proportion. Further, the no limiting amino acid sources were modeled from 10% up to 80%. Because no limiting amino acid sources inherently have a high protein quality, this ensures complementation between the sources.

Protein ratios and combinations for optimizing protein quality within the set nutritional constraints are displayed in Tables 4A, 4B, 5A, and 5B. Table 4B is a continuation of Table 4A, and Table 5B is a continuation of Table 5A. The model uncovered 3276 different mixes of ingredients for each ratio.

Tables 4A and 4B illustrate results from the model with the following macro nutrient constraints: energy (Kcal): 95-500, protein (%): 3.2-10 and dietary fiber (g): 1.5-5. The results indicate that the rows with the ratios of 10:60:30 (lysine limiting:methionine and cysteine limiting:no limiting), 10:50:40, 10:40:50, and 10:30:60 have the most optimal protein proportions with the corresponding nutrient profile and the optimal protein source mixes.

TABLE 4A
Proportion From Plant Protein Sources Meeting Macro Nutrient Constraints

| | | | | | | | Total | Total | Saturated | | | | |
|---|---|---|---|---|---|---|---|---|---|---|---|---|---|
| | Methionine | | Energy | Protein | Carb (% | Fibre | sugar (% | fat (% | fat (% | Iron | Calcium | Zinc | Sodium |
| Lysine | Cysteine | No | (Kcal) | (%) | energy) | (g) | energy) | energy) | energy) | (mg) | (mg) | (mg) | (mg) |
| Limiting | Limiting | Limiting | Mean | Mean | Mean | Mean | Mean | Mean | Mean | Mean | Mean | Mean | Mean |
| (%) | (%) | (%) | (SD) | (SD) | (SD) | (SD) | (SD) | (SD) | (SD) | (SD) | (SD) | (SD) | (SD) |
| 10 | 80 | 10 | 135.6 | 4.92 | 73.98 | 3.03 | 19.91 | 20.44 | 4.16 | 1.36 | 43.09 | 0.85 | 23.06 |
| | | | (46.6) | (1.27) | (10.59) | (0.74) | (20.47) | (12.52) | (4.55) | (0.56) | (25.31) | (0.34) | (21.1) |
| 10 | 70 | 20 | 135.1 | 5.91 | 69.32 | 3.09 | 19.94 | 23.18 | 4.23 | 1.69 | 57.76 | 0.91 | 24.41 |
| | | | (41.5) | (1.58) | (10.24) | (0.76) | (17.69) | (11.75) | (3.82) | (0.62) | (36.5) | (0.33) | (21.04) |
| 10 | 60 | 30 | 135.3 | 6.73 | 65.98 | 3.25 | 19.34 | 23.71 | 3.96 | 1.91 | 62.92 | 0.99 | 24.39 |
| | | | (35.49) | (2.08) | (10.53) | (0.83) | (15.05) | (11.06) | (3.01) | (0.72) | (47.55) | (0.36) | (20.77) |
| 10 | 50 | 40 | 133.8 | 6.43 | 66.33 | 3.51 | 16.87 | 20.55 | 3.42 | 1.91 | 43.17 | 1.02 | 27.84 |
| | | | (29.28) | (2.02) | (10.15) | (0.87) | (12.24) | (9.21) | (2.55) | (0.91) | (37.12) | (0.38) | (22.85) |
| 10 | 40 | 50 | 129.4 | 6.34 | 64.98 | 3.52 | 14.82 | 19.02 | 3.01 | 1.69 | 32.20 | 1.03 | 29.9 |
| | | | (22.81) | (1.61) | (11.51) | 0.9) | (9.51) | (7.82) | (2.05) | (0.73) | (17.93) | (0.41) | (21.83) |
| 10 | 30 | 60 | 129.1 | 6.19 | 66.39 | 3.51 | 12.44 | 17.82 | 2.74 | 1.67 | 28.92 | 1.03 | 28.72 |
| | | | (20.4) | (1.42) | (12.61) | (0.86) | (8.88) | (8.37) | (1.62) | (0.73) | (13.34) | (0.42) | (23.35) |
| 10 | 20 | 70 | 127.7 | 5.86 | 68.53 | 3.39 | 10.67 | 16.65 | 2.59 | 1.61 | 26.68 | 1.02 | 25.94 |
| | | | (19.96) | (1.44) | (13.95) | (0.85) | (9.02) | (9.07) | (1.51) | (0.74) | (13.63) | (0.44) | (22.78) |
| 10 | 10 | 80 | 128.9 | 6.44 | 70.02 | 3.44 | 6.68 | 13.57 | 2.41 | 1.99 | 26.18 | 1.26 | 23.74 |
| | | | (20.81) | (1.83) | (12.66) | (0.81) | (8.42) | (8.81) | (1.65) | (1.01) | (14.12) | (0.55) | (20.76) |

TABLE 4B
Proportion From Plant Protein Sources Meeting Macro Nutrient Constraints

| | Methionine | | | | | |
|---|---|---|---|---|---|---|
| Lysine | Cysteine | No | PDCAAS | Top | Error | |
| Limiting | Limiting | Limiting | median | solution | rate | |
| (%) | (%) | (%) | (IQR) | N (%) | N (%) | Examples of optimal mixes |
| 10 | 80 | 10 | 0.89 | 296 | 1792 | Missing beans/pulses |
| | | | (0.79, 0.99) | | (54.7%) | Cashew nuts - apples - soybeans |
| | | | | | | Almonds - potato - tofu |
| | | | | | | Oats - potato - soybeans |
| | | | | | | Cashew nuts - mushroom - tofu |
| | | | | | | Sunflower seeds - potatoes - yeast |
| 10 | 70 | 20 | 0.97 | 384 | 1896 | Apart from fava and kidney beans |
| | | | (0.83, 1.07) | | (57.8%) | missing other beans/pulses. |
| | | | | | | Sunflower seeds - fava bean - soya milk |
| | | | | | | Cashew nuts - carrots - cowpeas |
| | | | | | | Cashew nuts - kidney beans - soya milk |
| | | | | | | Oat - mushrooms - tofu |
| | | | | | | Oat - potatoes - tofu |
| 10 | 50 | 40 | 0.99 | 369 | 2140 | RTE cereals - apples - tofu |
| | | | (0.83, 1.07) | | (65.3%) | Sunflower seeds - chickpeas - soya milk |
| | | | | | | Oats - kidney beans - soya milk |
| | | | | | | White bread - potatoes - cowpeas |
| | | | | | | Coconut milk - split peas - soya milk |
| 10 | 50 | 40 | 0.99 | 369 | 2140 | RTE cereals - apples - tofu |
| | | | (0.83, 1.07) | | (65.3%) | Sunflower seeds - chickpeas - soya milk |
| | | | | | | Oats - kidney beans - soya milk |
| | | | | | | White bread - potatoes - cowpeas |
| | | | | | | Coconut milk - split peas - soya milk |
| 10 | 40 | 50 | 1 | 432 | 2321 | Oats - apples - cowpeas |
| | | | (0.87, 1.05) | | (70.8%) | Bread - lentils - soya milk |
| | | | | | | Oats - adzuki beans - soya milk |
| | | | | | | Barley - apple - soybeans |
| 10 | 30 | 60 | 0.99 | 543 | 2295 | Oat - adzuki beans - soya milk |
| | | | (0.88, 1.05) | | (70.1%) | Peanuts - chickpeas - soya milk |
| | | | | | | Walnuts - fava beans - cowpeas |
| | | | | | | Bread - potatoes - cowpeas |
| | | | | | | Tortilla - black beans - buckwheat |
| 10 | 20 | 70 | 0.97 | 626 | 2268 | Oats - potato - cowpeas |
| | | | (0.87, 1.04) | | (69.2%) | Cashew nuts - lentils - soy milk |
| | | | | | | Brown rice - lentils - buckwheat |
| | | | | | | Almonds - split peas - soy milk |
| | | | | | | Oats - chickpeas - soy milk |
| 10 | 10 | 80 | 1.02 | 952 | 2324 | Oats - apple - cowpeas |
| | | | (0.91, 1.08) | | (70.9%) | Cashew nuts - lentils - soy milk |
| | | | | | | Tortilla - split peas - buckwheat |
| | | | | | | Oats - navy beans - soya milk |
| | | | | | | Sunflower seeds - potato - soya milk |

Tables 5A and 5B illustrate results from the model with the following macro and micro nutrient constraints: energy (Kcal): 95-500, protein (%): 3.2-10, dietary fiber (g): 1.5-5, iron (mg): 0.9-2.8, calcium (mg): 79-250 and zinc (mg): 0.1-1.2. The results indicate that the row with the ratio of 50:10:40 (lysine limiting: methionine and cysteine limiting: no limiting) has the most optimal protein proportions with the corresponding nutrient profile and the optimal protein source mixes.

TABLE 5A
Proportion from Plant Protein Sources Meeting Macro and Micro Nutrient Constraints

| | | | | | | | Total | Total | Saturated | | | | |
|---|---|---|---|---|---|---|---|---|---|---|---|---|---|
| | Methionine | | Energy | Protein | Carb (% | Fibre | sugar (% | fat (% | fat (% | Iron | Calcium | Zinc | Sodium |
| Lysine | Cysteine | No | (Kcal) | (%) | energy) | (g) | energy) | energy) | energy) | (mg) | (mg) | (mg) | (mg) |
| limiting | limiting | limiting | Mean | Mean | Mean | Mean | Mean | Mean | Mean | Mean | Mean | Mean | Mean |
| (%) | (%) | (%) | (SD) | (SD) | (SD) | (SD) | (SD) | (SD) | (SD) | (SD) | (SD) | (SD) | (SD) |
| 10 | 80 | 10 | 113.5 | 5.27 | 67.13 | 3.20 | 17.05 | 35.92 | 5.17 | 1.51 | 98.85 | 0.74 | 24.41 |
| | | | (32.22) | (0.65) | (7.36) | (0.73) | (9.77) | (13.04) | (2.85) | (0.29) | (11.68) | (0.16) | (12.87) |
| 10 | 70 | 20 | 133.7 | 6.79 | 65.76 | 2.94 | 20.18 | 28.18 | 4.62 | 1.78 | 98.64 | 0.80 | 23.69 |
| | | | (38.59) | (1.04) | (8.57) | (0.66) | (17.49) | (11.37) | (3.41) | (0.34) | (20.04) | (0.17) | (19.10) |
| 10 | 60 | 30 | 135.8 | 8.49 | 59.34 | 3.01 | 19.91 | 32.21 | 4.76 | 2.2 | 127.78 | 0.87 | 20.77 |
| | | | (37.83) | (1.09) | (7.01) | (0.83) | (14.77) | (9.19) | (2.43) | (0.29) | (25.16) | (0.17) | (17.16) |
| 50 | 10 | 40 | 164.4 | 9.5 | 61.73 | 2.85 | 3.16 | 33.01 | 5.20 | 2.35 | 156.21 | 1.13 | 8.48 |
| | | | (24.18) | (0.33) | (7.29) | (0.48) | (2.08) | (4.41) | (0.89) | (0.14) | (2.66) | (0.03) | (2.07) |
| 21 | 29 | 50 | 199.1 | 7.34 | 60.81 | 4.39 | 12.2 | 27.18 | 3.34 | 1.44 | 78.92 | 1.04 | 15.73 |
| | | | (23.01) | (1.05) | (13.24) | (0.28) | (8.76) | (6.08) | (1.89) | (0.24) | (0.53) | (0.05) | (11.06) |
| 18 | 22 | 60 | 162.1 | 6.56 | 47.25 | 3.25 | 22.75 | 35.13 | 3.86 | 1.23 | 79.17 | 0.76 | 34.25 |
| | | | (15.94) | (0.72) | (2.66) | (0.89) | (5.95) | (3.48) | (1.31) | (0.18) | (0.78) | (0.08) | (3.66) |
| 17 | 13 | 70 | 157.4 | 6.89 | 44.52 | 2.98 | 22.31 | 35.05 | 3.72 | 1.23 | 79.05 | 0.75 | 38.69 |
| | | | (6.23) | (0.93) | (1.38) | (0.35) | (1.58) | (1.35) | (0.67) | (0.12) | (1.10) | (0.06) | (2.81) |
| 10 | 10 | 80 | 103.8 | 5.08 | 47.99 | 4.54 | 24.70 | 29.70 | 3.51 | 1.44 | 86.03 | 0.64 | 43.31 |
| | | | (4.11) | (0.59) | (1.44) | (0.27) | (1.85) | (0.9) | (0.38) | (0.08) | (1.77) | (0.04) | (1.68) |

TABLE 5B
Proportion from Plant Protein Sources Meeting Macro and Micro Nutrient Constraints

| | Methionine | | | | | |
|---|---|---|---|---|---|---|
| Lysine | Cysteine | No | PDCAAS | Top | Error | |
| Limiting | Limiting | Limiting | median | solution | rate | |
| (%) | (%) | (%) | (IQR) | N (%) | N (%) | Examples of optimal mixes |
| 10 | 80 | 10 | 0.89 | 22 | 3135 | Missing beans/pulses |
| | | | (0.83, 0.97) | | (95.7%) | Walnuts - kale - tofu |
| | | | | | | Cashew nut - kale - tofu |
| | | | | | | Oats - potato - soybeans |
| | | | | | | Cashew nut butter - kale - tofu |
| | | | | | | Almond butter - carrots - tofu |
| 10 | 70 | 20 | 1.07 | 114 | 2912 | Missing beans/pulses |
| | | | (0.95, 1.16) | | (88.8%) | Cashew nuts - potatoes - tofu |
| | | | | | | Bread - mushrooms - tofu |
| | | | | | | Walnuts - kale - soybeans |
| | | | | | | Oat - potatoes - tofu |
| | | | | | | Oat - apples - tofup |
| 10 | 60 | 30 | 1.11 | 90 | 2974 | Missing beans/pulses |
| | | | (1.01, 1.28) | | (90.7%) | Oats - potatoes - tofu |
| | | | | | | Sunflower seeds - mushrooms - tofu |
| | | | | | | Oats - apples - tofu |
| | | | | | | Peanuts - kale - soybean |
| | | | | | | Almond butter - kale - tofup |
| 50 | 10 | 40 | 1.12 | 26 | 3165 | Oat bran - kidney beans - tofu |
| | | | (1.1, 1.13) | | (96.6%) | Brown rice - potatoes - tofu |
| | | | | | | Oat bran - fava beans - tofu |
| | | | | | | White rice - pinto beans - tofu |
| | | | | | | White rice - chickpeas - tofu |
| 21 | 29 | 50 | 0.67 | 5 | 3235 | No optimal mix meeting PDCAAS 1 |
| | | | (0.65, 0.93) | | (98.7%) | |
| 18 | 22 | 60 | 0.98 | 7 | 3228 | No other optimal mix meeting |
| | | | (0.96, 0.98) | | (98.5%) | PDCAAS 1 |
| | | | | | | Almond butter - split peas - soya milk |
| 17 | 13 | 70 | 1.01 | 9 | 3249 | Almond butter - lentils - soya milk |
| | | | (0.99, 1.01) | | (99.1%) | Almond butter - split peas - soya milk |
| | | | | | | Almond butter - potato - soya milk |
| | | | | | | Almond butter - adzuki beans - soya milk |
| | | | | | | Almond butter - carrots - soya milk |
| 10 | 10 | 80 | 0.86 | 16 | 3260 | No optimal mix meeting PDCAAS 1 |
| | | | (0.85, 0.87) | | | |

1: A method of manufacturing a composition having optimal protein quality, the method comprising:
determining a ratio of a first protein source to a second protein source, wherein determining the ratio comprises:
receiving a first list of ingredients for the first protein source and receiving a second list of ingredients for the second protein source,
computing a plurality of combinations of the ingredients from the first list of ingredients with the ingredients from the second list of ingredients,
computing a protein quality score for each of the plurality of combinations at a plurality of ratios, and
identifying an optimal ratio of the first protein source to the second protein source, wherein the optimal ratio is a ratio of the first protein source to the second protein source meeting or exceeding a predefined protein quality score; and
combining the first protein source and the second protein source at the optimal ratio to thereby form at least a portion of the composition.

2: The method of claim 1, wherein the predefined protein quality score met or exceeded by the optimal ratio is a PDCAAS score of about 1.

3: The method of claim 1, wherein the first protein source comprises a limiting amino acid; and the second protein source comprises a limiting amino acid different than the limiting amino acid of the first protein source.

4: The method of claim 1, further comprising providing the composition to an individual consuming a plant based diet.

5: A composition made by the method of claim 1.

6: A composition comprising a combination of a first protein source and a second protein source, wherein the first protein source is selected from the group consisting of a protein with a lysine limiting amino acid and a protein with no limiting amino acid, and wherein the second protein source is selected from the group consisting of a protein with a lysine limiting amino acid, a protein with methionine and cysteine limiting amino acid, and a protein with no limiting amino acid.

7: The composition according to claim 6, wherein the first protein source comprises a protein with a lysine limiting amino acid, and wherein the protein with the lysine limiting amino acid comprises at least one protein from one or more of cereals, nuts, or seeds.

8: The composition according to claim 6, wherein the second protein source comprises a protein with a methionine and cysteine limiting amino acid, and wherein the protein with the methionine and cysteine limiting amino acid comprises at least one protein from legumes.

9: The composition according to claim 6, wherein the first protein source is at least 20% of the combination of the first protein source and the second protein source.

10: The composition according to claim 6, wherein the first protein source is plant based and comprises a protein with no limiting amino acids.

11: The composition according to claim 6, further comprising a third protein source, wherein the third protein source is selected from the group consisting of a protein with a lysine limiting amino acid, a protein with methionine and cysteine limiting amino acid, and a protein with no limiting amino acid, and wherein the third protein source is different protein from the first and second protein sources.

12: The composition of claim 10, wherein the third protein source comprises a protein with no limiting amino acid.

13: A method of increasing at least one of protein absorption, protein digestibility, and protein bioavailability in a diet of an individual, the method comprising administering a composition to the individual, the composition comprising a combination of protein sources, the combination comprising a first protein source and a second protein source, wherein the first protein source is selected from the group consisting of a protein with a lysine limiting amino acid and a protein with no limiting amino acid, and wherein the second protein source is selected from the group consisting of a protein with a lysine limiting amino acid, a protein with methionine and cysteine limiting amino acid, and a protein with no limiting amino acid, and wherein the first protein source is at least 10% of the combination of the protein sources.

14: The method of claim 12, wherein the diet of the individual is a plant based diet.

15: The method of claim 12, wherein the composition is orally administered to the individual.

16: The method of claim 12, wherein the composition further comprises at least one of carbohydrates, fats, vitamins, minerals or a third protein source.

17: A composition comprising a protein source, the protein source comprising a combination of:
at least 10% of a protein with a lysine limiting amino acid,
10-60% of a protein with methionine and cysteine limiting amino acids, and
30-80% of a plant-based protein with no limiting amino acid,
wherein the protein source comprises a PDCAAS score of about 1.

18: The composition of claim 17, wherein the protein source comprises 95-300 kcal.

19: The composition of claim 17, wherein the protein source comprises 3.2-10% protein.

20: The composition of claim 17, wherein the protein source comprises 1.5-5 grams of fiber.

21: The composition of claim 17, wherein the protein source comprises 0.9-2.8 mg iron.

22: The composition of claim 17, wherein the protein source comprises 79-250 mg calcium.

23: The composition of claim 17, wherein the protein source comprises 0.1-1.2 mg zinc.

24: The composition of claim 17, wherein the protein with the lysine limiting amino acid is selected from the group consisting of oat, barley, seitan, pumpkin seeds, white rice, and combinations thereof.

25: The composition of claim 17, wherein the protein with methionine and cysteine limiting amino acids is selected from the group consisting of kidney bean, potato, lentils, peas split, fava bean, black beans, peas, and combinations thereof.

26: The composition of claim 17, wherein the plant-based protein with no limiting amino acid is selected from the group consisting of soy milk, buckwheat, tofu, and combinations thereof.

27: The composition of claim 17, wherein the protein source comprises 50-60% of the protein with methionine and cysteine limiting amino acids.

28: The composition of claim 17, wherein the protein source comprises 30-40% of the plant-based protein with no limiting amino acid.

29: The composition of claim 17, wherein the protein with lysine as the limiting amino acid, the protein with methionine and cysteine as the limiting amino acids, and the protein without any limiting amino acids are the only protein sources in the composition.

30: The composition of claim 17, wherein all of the protein sources in the composition are plant based.

31: The composition of claim 17, further comprising an animal protein source.
